(19) United States
(12) Patent Application Publication
(10) Pub. No.: US 2022/0369907 A1
(43) Pub. Date: Nov. 24, 2022

(54) SCANNING SYSTEM FOR DETERMINING A HEALTH-CONDITION

(71) Applicant: 3SHAPE A/S, Copenhagen K (DK)

(72) Inventors: Nikolaj DEICHMANN, Klagshamn (SE); Esben Rosenlund HANSEN, Brønshøj (DK)

(73) Assignee: 3SHAPE A/S, Copenhagen K (DK)

(21) Appl. No.: 17/767,151

(22) PCT Filed: Oct. 9, 2020
(86) PCT No.: PCT/EP2020/078405

§ 371 Date: Apr. 7, 2022

(30) Foreign Application Priority Data
Oct. 10, 2019 (EP) 19202470.1

(51) Int. Cl. A61B 1/00; A61B 1/06; A61B 1/247

(52) U.S. Cl. CPC A61B 1/000096; A61B 1/00172; A61B 1/00194; A61B 1/0655; A61B 1/247; A61B 1/00045

(57) ABSTRACT

Disclosed is a scanning system for determining a health-condition based on scanning an intraoral object. More specifically, the present disclosure is related to different methods of providing the health-condition, for example by adapting the scanner with different scanning modes.

### FIELD OF THE INVENTION

The present disclosure relates to a scanning system for determining a health-condition based on scanning an intraoral object. More specifically, the present disclosure is related to different methods of providing the health-condition, for example by adapting the scanner with different scanning modes.

### BACKGROUND OF THE INVENTION

Recording images using different illumination-modes is known in the field of dental healthcare. For example, 3D-scanners are known to be configured for illuminating a tooth using both white light, for example from a white LED, and light of different color, for example from a red laser diode.

In some 3D-scanners, 2D-images are thus acquired with in two separate modes with white-light illumination and with red-light illumination.

The 2D-images acquired with the red-light illumination may be used for forming a 3D-representation of a tooth, and the 2D-images acquired with the white light may be used for obtaining colors of the tooth. The colors from the 2D-images are associated to the surface of the 3D-representation, for example by a color-mapping algorithm.

Thus, in presently known 3D-scanners used in the dental healthcare, color-mapping is related to mapping of colors from 2D-images to the outer surface of the 3D-representation of for example a tooth. The color of the outer surface of a tooth represents the actual color of the tooth. By knowing the color of a tooth, that color may be matched to the color of a dental restoration, such as a crown, an inlay, an onlay and/or a bridge. Further, by knowing the color, one can distinguish between teeth and gingiva. Thus, color images of for example teeth are important not only for visualization of teeth but also for dental restorations.

Today, imaging and scanning of the outside surface of for example a tooth is well-established and can be achieved using various imaging techniques.

Further, bacteria and tooth decay can be imaged using blue, violet or ultraviolet (UV) light, causing the bacteria and tooth material to fluoresce. The 2D-images acquired with UV-light is also known to be correlated to the outer surface of for example a tooth. Thus, an overlay of a 3D structure with fluorescence information is known in the art.

Even further, the inside of a tooth, i.e. approximal caries lesions in enamel, can be imaged using infrared (IR) light. More specifically, a 3D-representation of the inside of a tooth can be obtained by acquiring a set of 2D-images during IR-light illumination. The 3D-IR-representation of the inside of the tooth may be correlated to the outside of the tooth by various techniques. For example, the correlation of a 3D-IR-representation to a 3D-representation recorded using white light or red light can be achieved by matching features in the two representations.

Thus today, correlation of one 3D-representation obtained in one illumination-mode within another 3D-representation obtained in a second illumination-mode is known in general.

Accordingly, a scanning device that changes between illumination-modes is known in general. Further, a scanning device that has different illumination-modes can be used to determine a region of interest inside a mouth, for example showing different types of carries or other types of dental conditions. More specifically, IR-light illumination and UV-light illumination can be used to determine degrees of carries and thereby provide a region of interest if the degree is above a specific threshold.

Changing between illumination modes is mostly fixed in the scanning device. For example, the scanning device may be fixed to first operate a first light source in a first period and then to operate a second light source in a second period. However, some scanning devices are also known to adapt the illumination-mode, for example according to additional measurements being made in an intra-oral cavity, for example where the additional measurement is related to image quality of acquired images.

For scanning devices that may determine a region of interest of an intraoral object, adaptive scanning is not yet explored in detail.

As an example, US 2019/0231491 discloses a scanning system that may determine a region of interest and at the same time may also adapt a scanning device according to the region of interest. In US 2019/0231491, the adaption is more specifically related to markers (i.e. a pin, flag) of the region of interest, where the markers may have notes associated with it. As described in US 2019/0231491, the markers may modify the manner in which later scans are taken. Further, US 2019/0231491 discloses that later scans may dynamically be modified by the flags from earlier scans (one day or more) or by a detection of a change in region (even unmarked regions) compared to earlier scans. In other words, US 2019/0231491 discloses how data that is older than one day is used to adapt the scanning of the scanning device. The reason why old data is used to adapt the scanning of the scanning device is simply because the adaption is based on changes that can only occur over one day or more. However, adaption based on changes that can only occur over one day or more is not optimal. There is thus a need for providing a scanning system with a more optimal adaption.

All in all, an optimized adaptive scanning device that determines diagnostic feature(s) needs to be explored in more details.

### SUMMARY OF THE INVENTION

An objective of the present disclosure is to explore adaptive scanning devices in more details. Further, an objective of the present invention is to provide a scanning device that efficiently adapts to the scanning environment.

The present disclosure provides an adaptive scanning device for determining a health-condition and/or a probability thereof. More specifically, the present disclosure provides an adaptive scanning system for determining a health-condition and/or a probability thereof based on scanning an intraoral object, for example of at least a part of a tooth and/or a at least a part of gingiva. Accordingly, a 3D-model as obtained by scanning the intraoral object according to the present disclosure may be defined to be a part of a 3D-model, for example a part that relates to at least a part of a tooth and/or a at least a part of gingiva.

The scanning system of the present disclosure comprises a scanning device comprising: an illumination-unit configured to illuminate the intraoral object with light; an image-sensor configured to record images of light from the illuminated intraoral object; an illumination-controller configured to operate the illumination-unit in a first illumination-mode, and configured to operate the illumination unit in a second illumination-mode, and/or an acquisition-controller configured to operate the image-sensor in a first acquisition-mode, and configured to operate the image-sensor in a second acquisition-mode.

The scanning device is configured to change between the first illumination-mode and the second illumination-mode, whereby the scanning device forms a first dataset of the intraoral object when in the first illumination-mode and whereby the scanning device forms a second dataset of the intraoral object when in the second illumination-mode.

Changing between the first illumination-mode and the second illumination-mode results in a scanning device where a scanning of two different types of can be made in one scan without the need of changing between illumination modes in two separate scans or scan sessions.

Alternatively or additionally, the scanning device is configured to change between the first acquisition-mode and the second acquisition-mode, whereby the scanning device forms a first dataset of the intraoral object when in the first acquisition-mode and whereby the scanning device forms a second dataset of the intraoral object when in the second acquisition-mode.

The change between said modes is understood to be automatic.

The adaptive scanning system further comprises a data processor configured to: form, from the first dataset, a 3D-model of the intraoral object; form, from the second dataset, a 2D-image of the intraoral object and/or further details of the 3D-model; apply, on the 2D-image and/or the 3D-model, a diagnostic algorithm to identify a diagnostic feature of the intraoral object, determine, based on the diagnostic feature of the intraoral object, the health-condition and/or the probability thereof.

The herein defined health-condition and/or probability thereof is different from a region of interest. The diagnostic feature may be a region of interest. The herein defined health-condition and/or probability thereof may be an analysis (defined by the diagnostic algorithm) of the diagnostic feature, for example a region of interest, which may result in a tangible understanding of which disease, lesion, and/or condition that is associated with the diagnostic feature or region of interest. For example, the health-condition may be information such as “interproximal carries” and/or the probability of the health-condition may be “92% probability of interproximal carries”. The 3D-model and the health-condition may be displayed in for example a user-interface.

In a first perspective of the invention, the data processor is further configured to redefine, during the scan where the diagnostic feature is determined and based on the determined health-condition and/or the probability thereof, the first illumination-mode and the second illumination-mode, and/or redefine, during the scan where the diagnostic feature is determined and based on the determined health-condition and/or the probability thereof, the first acquisition-mode and the second acquisition-mode.

Accordingly, the present invention as defined in this first aspect differs from US 2019/0231491 in that the scanner as described herein changes the illumination-mode and/or acquisition-mode during the scan where the diagnostic feature is determined, i.e. during the present scan, and/or during the scan as made of the intraoral object, and/or in real-time or in close proximity thereof.

A scan may be defined as the period of time that is used to provide a 3D-model of the intraoral object(s) in an intraoral cavity. A scan therefore lasts between 5 seconds and maybe up to 1 minute, or up several minutes if the intraoral object(s) are re-scanned several times during the scan. Alternatively or additionally, a scan may be a full arch scan of the maxilla and mandibular. A scan is to be understood as the time the intraoral scanning device is inside the intraoral cavity during a continuous session constructing the desired 3D model. A typical scan (even with several re-scans, pre-scans or prep-scans) typically does not take more than 30 minutes. Accordingly, the present invention may change the illumination-mode and/or acquisition-mode in a scan as defined to last between 5 seconds and 30 minutes. According to the invention, a scan as herein defined has a duration of less than a day, and therefore does not provide data that is older than one day.

If the same intra-oral object is re-scanned on the same day using the scanning system according the present invention, the illumination-mode and/or the acquisition mode may change according to the invention. Therefore, it is also possible with the present invention that the health-condition and/or the probability thereof changes or updates on the same day and/or during the scan. For example, this may be the case when extensive caries has been detected and the dentist (and patient) has agreed to repair the damaged tooth. An initial scan may, using the present invention, reveal the severe caries lesion. During a process of removing any infected tooth material, for example using drilling, scans or scan sessions may be performed in-between drilling sessions. The scans may provide an updated health condition, for example a score of the apparent dental condition, such that the dentist only removes the correct amount of damaged tooth and not healthy material.

This provides a more accurate health-condition and/or a more accurate health-probability. Preferably, the more times the intra-oral object is re-scanned during the scan on the day, the more accurate the health-condition and/or the more accurate the probability thereof. Most preferably, the health-condition and/or the probability thereof converge(s) to a single health-condition and/or a single probability during the scan.

The present invention as defined in the first aspect provides a real-time health-condition and/or probability thereof which may adapt during scanning.

The health-condition and/or probability thereof is in one embodiment not necessarily determined only from dataset(s) that is/are formed during a scan, and/or the scan. In some embodiments, the health-condition and/or probability thereof may partly be based on dataset(s) that is/are different from the data as formed during the scan. In some embodiments, the health-condition and/or probability thereof may partly be based on dataset(s) that is/are older than 24 hours. However, regardless of how the health-condition and/or probability thereof is determined, the scanning device adapts during the scan where the diagnostic feature is determined.

In another embodiment, the determination of the health-condition and/or the probability thereof is independent of one or more dataset(s) of the intraoral object that is/are formed 24 hours or more before the first dataset and second dataset being formed. In this embodiment, the health-condition and/or the probability thereof does not rely on comparative analysis with old data, i.e. data that is formed 24 hours or more before the first dataset and second dataset being formed, of the same tooth, but instead is able to analyse the first and/or the second dataset in a more direct or absolute manner. For example, in one embodiment, the health-condition and/or the probability thereof is based on machine learning and/or artificial intelligence. Methods like these may not need to rely on dataset(s) of the same tooth, but the method may instead rely on learning-algorithms operated on a training-set of teeth. Whereas the scanner as disclosed US 2019/0231491 changes scanning parameters based on previous scans of an intraoral object, i.e. scan data of the intraoral object that is older than one day, the present embodiment of the scanning system changes scanning parameters based only on the current scan. By not relying on old data, the present embodiment of the invention is able to firstly provide the health-condition and/or the probability thereof of a patient coming into a clinic where no data of the patient exists.

As previously described, the present invention as defined by the first aspect is able to adapt the scanning device according to the patient and/or the patient's health-condition(s) and/or probabilities thereof during scanning (during a scan where the diagnostic feature is determined), whereby the health-condition or the probability thereof may be optimized. Accordingly, the present invention as defined by the first aspect provides an optimized determination of a health-condition and/or of the probability thereof.

The adaptive scanning system according to the first aspect further comprises a display, whereon the 3D-model and the health-condition and/or probability thereof are displayed.

In a second aspect of the invention, the data processor is further configured to define, based on a diagnostic training-feature of an intraoral object that differs from the diagnostic feature of the intraoral object, the first illumination-mode and the second illumination-mode, and/or define, based on a diagnostic training-feature of an intraoral object that differs from the diagnostic feature of the intraoral object, the first acquisition-mode and the second acquisition-mode.

Accordingly, the present invention as defined in the second aspect differs from US 2019/0231491 in that the scanner as described herein defines said modes based on a diagnostic training-feature of an intraoral object that differs from the diagnostic feature of the intraoral object.

For example, the intraoral object to define said modes may in one embodiment be a test object with known health-conditions(s) and/or probabilities thereof.

Additionally, or alternatively, the intraoral object to define said modes may in another embodiment be a plurality of test objects from various patients with various health-conditions(s) and/or probabilities thereof. In this context, said modes may be found as an optimum based on analyzing scan data from various patients having various health-conditions and/or probabilities thereof. The analysis of the scan data may be based on clinical evaluation, machine learning and/or artificial intelligence.

The adaptive scanning system according to the second aspect further comprises a display, whereon the 3D-model and the health-condition and/or probability thereof are displayed.

The second aspect differs from the first aspect in that said modes are pre-defined, and do not necessarily change during scanning. However, a combination of the first aspect and the second aspect is possible. The combination of the first aspect and the second aspect is provided as the third aspect described below.

The third aspect of the invention provides a scanning system for determining a health-condition and/or a probability thereof based on scanning of an intraoral object, the scanning system comprising: a scanning device to scan the intraoral object, comprising: an illumination-unit configured to illuminate the intraoral object with light; an image-sensor configured to record images of light from the illuminated intraoral object; an illumination-controller configured to operate the illumination-unit in a first illumination-mode, and configured to operate the illumination unit in a second illumination-mode, and/or an acquisition-controller configured to operate the image-sensor in a first acquisition-mode, and configured to operate the image-sensor in a second acquisition-mode, wherein the scanning device is configured to change between the first illumination-mode and the second illumination-mode, whereby the scanning device forms a first dataset of the intraoral object when in the first illumination-mode and whereby the scanning device forms a second dataset of the intraoral object when in the second illumination-mode, and/or wherein the scanning device is configured to change between the first acquisition-mode and the second acquisition-mode, whereby the scanning device forms a first dataset of the intraoral object when in the first acquisition-mode and whereby the scanning device forms a second dataset of the intraoral object when in the second acquisition-mode; a data processor configured to: form, from the first dataset, a 3D-model of the intraoral object; form, from the second dataset, a 2D-image of the intraoral object and/or further details of the 3D-model; apply, on the 2D-image and/or the 3D-model, a diagnostic algorithm to identify a diagnostic feature of the intraoral object, determine, based on the diagnostic feature of the intraoral object, the health-condition and/or probability thereof, redefine, during the scan where the diagnostic feature is determined and based on the diagnostic feature or the related determined health-condition and/or the probability thereof, the first illumination-mode and the second illumination-mode, and/or redefine, during the scan where the diagnostic feature is determined and based on the diagnostic feature or the related determined health-condition and/or the probability thereof, the first acquisition-mode and the second acquisition-mode, and/or define, based on a diagnostic training-feature of an intraoral object that differs from the diagnostic feature of the intraoral object, the first illumination-mode and the second illumination-mode, and/or define, based on a diagnostic training-feature of an intraoral object that differs from the diagnostic feature of the intraoral object, the first acquisition-mode and the second acquisition-mode; and a display, whereon the 3D-model and the health-condition and/or probability thereof are displayed.

In a fourth aspect of the invention, the present disclosure provides a scanning system for determining a health-condition and/or a probability thereof based on scanning of an intraoral object, the scanning system comprising: a scanning device to scan the intraoral object, comprising: an illumination-unit configured to illuminate the intraoral object with light; an image-sensor configured to record images of light from the illuminated intraoral object; an illumination-controller configured to operate the illumination-unit in a first illumination-mode, and configured to operate the illumination unit in a second illumination-mode; and an acquisition-controller configured to operate the image-sensor in a first acquisition-mode, wherein the first acquisition-mode is defined by a first gain-value, and configured to operate the image-sensor in a second acquisition-mode, wherein the second acquisition-mode is defined by a second gain-value, wherein the scanning device is configured to change between the first illumination-mode and the second illumination-mode, whereby the scanning device forms a first dataset of the intraoral object when in the first illumination-mode and whereby the scanning device forms a second dataset of the intraoral object when in the second illumination-mode, and wherein the scanning device is further configured to change between the first acquisition-mode and the second acquisition-mode, whereby the scanning device forms the first dataset of the intraoral object when in the first acquisition-mode and whereby the scanning device forms the second dataset of the intraoral object when in the second acquisition-mode; a data processor configured to: form, from the first dataset, a 3D-model of the intraoral object; form, from the second dataset, a 2D-image of the intraoral object and/or further details of the 3D-model; apply, on the 2D-image and/or the 3D-model, a diagnostic algorithm to identify a diagnostic feature of the intraoral object, determine, based on the diagnostic feature of the intraoral object, the health-condition and/or probability thereof, a display, whereon the 3D-model and the health-condition and/or probability thereof are displayed.

Accordingly, the present invention as defined in the fourth aspect differs from US 2019/0231491 in that the scanner as described herein provide that said acquisition-modes are defined by gain-values.

The scanning device as provided in the fourth aspect thus changes gain-values from when the scanning device forms the first dataset of the intraoral object when to when the scanning device forms the second dataset. As previously described, the first dataset forms a 3D-model, or a part of a 3D-model, and therefore the first dataset may in general be a single 2D-image or a part thereof. Accordingly, the present invention as defined by the fourth aspect, is thus able to change gain values between two types of dataset, where the one type of dataset is used to form a 3D-model in the first illumination mode, for example acquired with white-light illumination, and the second type of dataset is a part of an additional 2D-image in the second illumination-mode, for example acquired with fluorescence-light illumination used to form additional details of the 3D-model and/or used in the determination of the health-condition and/or the probability thereof.

Changing the gain-values between individual 2D-images having different illumination-modes as recorded during scanning provides that the different types of images may have similar intensity, visibility or brightness. For example, an image acquired during white-light illumination, may have a very high visibility or high brightness, and if another image acquired during fluorescence-light illumination, that other image may have very low visibility. By setting the gain-value differently when the two different images are acquired, the visibility of the two different images may become similar. By not using the same gain for two different images, for example a white-light image, and a fluorescence-light image, this also has the advantage of avoiding necessary amplification of the white-light image, which therefore avoids a noisy white-light image.

In relation to noise, it is known in the field of imaging, that increasing gain, also increases noise. It is thus known in the filed of imaging, that gain should only be used as a last resort to increase brightness. Instead, as is known in the field of imaging, brightness should be increased by other means, such as increasing the intensity of the light source, and/or such as increasing the integration/exposure time.

However, the inventor of the present invention as defined by the fourth aspect has realized, in contradiction to the above understanding, that gain should preferably be used in a scanning device to scan intraoral object(s). The reason is firstly that increasing the gain provides a scanning device that consumes much less power in comparison to increasing the intensity of the light source. Secondly, increasing gain also provides a scanning device that can provide more information in less time than in comparison to increasing the integration/exposure time (as this obviously increases acquisition time). The inventors have therefore by the fourth aspect provided a scanning device that is both power efficient and time efficient.

### BRIEF DESCRIPTION OF THE DRAWINGS

The above and/or additional objects, features and advantages of the present invention, will be further described by the following illustrative and non-limiting detailed description of embodiments of the present invention, with reference to the appended drawing(s), wherein:

FIG. 1 shows an example of a scanning system according to the invention.

FIG. 2 shows an example of a scanning system according to the first aspect of the invention.

FIG. 3 shows an example of a scanning system according to the fourth aspect of the invention.

FIG. 4 shows an example how of the 3D-model and the health-condition is displayed in a user interface on a display.

### DETAILED DESCRIPTION

### The 2D-Image

In one embodiment, the data processor is further configured to correlate at least a part of the 2D-image to at least a corresponding 3D-point on or inside the 3D-model, whereby the at least part of the 2D-image and the health-condition and/or the probability thereof is related to a 3D-location of the 3D-point on or inside the 3D-model.

In another embodiment, the 2D-image or the at least part of the 2D-image with the diagnostic feature is displayed with a 2D-3D indicator between the 2D-image or the at least part of the 2D-image and the 3D-model to show how the 2D-feature correlates to the 3D-location of the 3D-point on or inside the 3D-model. For example, the indicator may be an arrow or a line. Further, when displayed as described above, the 2D-image is further displayed on the display, in this particular embodiment, separately from the 3D-model. By separately displaying a 2D-image, the operator of the scanning device is presented with the 3D-model, the health-condition and/or the probability thereof together with a visual 2D presentation of the diagnostic feature. In this manner, the operator is presented with the diagnostic feature faster than if it was only presented on the 3D-model, for example because the 3D-model would need to be rotated to see the diagnostic feature.

In most embodiments, the 2D-image or part(s) thereof is/are used to form further details of the 3D-model. For example, the 2D-image may provide fluorescence texture to the 3D-model, or the 3D-image may provide high resolution color and/or texture to the 3D-model. In more preferred embodiments, the 2D-image is a fluorescent 2D-image, an infrared 2D-image, or a high-resolution 2D-image.

The Health-Condition and/or the Probability Thereof

In one embodiment, the health-condition and/or the probability thereof is displayed with a 3D-diagnosis indicator between the health-condition and/or the probability thereof and the 3D-model to show how the health-condition and/or the probability thereof correlates to the 3D-location of the 3D-point on or inside the 3D-model. For example, the indicator may be a symbol, an arrow or a line.

In another embodiment, the health-condition and/or the probability thereof is displayed with a 2D-diagnosis indicator between the health-condition and/or the probability thereof and the 2D-image or the at least part of the 2D-image to show how the health-condition and/or probability thereof correlates to the diagnostic feature.

In most embodiments, the health-condition and/or the probability thereof is related to carries and/or bacteria. However, in some embodiments, the health-condition and/or the probability thereof is related to cancer. Cancer in general may in some embodiments be identified in the intraoral cavity.

### The Diagnostic Algorithm

In a preferred embodiment, the diagnostic algorithm is based on artificial intelligence and/or is based on pattern recognition. As previously described, this embodiment provides that the health-condition need not to be dependent on previous scans of a given patient.

### The Illumination-Modes and Acquisition-Modes

In one embodiment, said first illumination mode is white-light-illumination, and wherein said second illumination mode is fluorescence-light-illumination.

In another embodiment said first illumination mode is white-light-illumination, and wherein said second illumination mode is infrared-light-illumination.

In yet another embodiment, the scanning device has more than two illumination-modes, for example three illumination-modes, for example a white-light-illumination mode, a fluorescence-light-illumination mode, and an infrared illumination mode. The more illumination-modes, the more heath-conditions may be determined. For example, using both fluorescence-light-illumination mode and an infrared illumination mode may provide both surface related carries and sub-surface related carries.

In one embodiment, said first acquisition-mode is defined by a low spatial resolution and said second acquisition-mode is defined by a high spatial resolution.

In a preferred embodiment, said first acquisition-mode is defined by a first gain-value and said second acquisition-mode is defined by a second gain-value.

Gain is typically adjusted before the pixel output (in voltage, provided by converting charge to voltage using a capacitor circuit) is converted to a digital signal using an analog/digital (ND) converter. In other words, gain amplifies the analog signal from pixel(s) before conversion. Together with gain, the offset can also be adjusted. By increasing gain, one increases the gray-value output from the detector, which therefore provides a brighter image.

In a most preferred embodiment, the first gain-value and the second gain-value are controlled via a pin to the image sensor, whereby the first gain-value is synchronized with the first dataset or parts thereof, and the second gain-value is synchronized with the second dataset or parts thereof or the 2D-image or parts thereof. For example, the image sensor may be a CMOS sensor. The entire CMOS image sensor may be comprised in an integrated circuit package and may be placed on printed circuit boards (PCBs). The circuit components on the CMOS image sensor may all be comprised within this package, and according to the just described embodiment, at least a pin, more specifically an external pin or more external pins may be used to access and control the image sensor, the control may for example be from a Field-Programmable Gate Array (FPGA) processor. Most preferably, a single pin is used to control the gain. As previously described, the first dataset to form a 3D-model may be a plurality of 2D-images. Accordingly, all the plurality of 2D-images or parts thereof used to form the 3D-model may in some embodiments be individually synchronized with the first gain-value. The synchronization may be in relation to the exposure time as used to acquire the first dataset or parts thereof and as used to acquire the second dataset of parts thereof. By controlling the gain using a pin to the image sensor, the gain-values can be controlled in a fast and adaptive manner.

In another preferred embodiment, said first illumination-mode is defined by a first period of illumination-time and said second illumination-mode is defined by a second period of illumination-time. Examples of illumination-times for each mode may be in the order of milliseconds. Acquisition of for example 200 images, each acquired with a different mode, may for example be acquired as a focus-stack by moving a lens.

In yet another preferred embodiment, said first acquisition-mode is defined by a first period of acquisition-time and/or said second acquisition-mode is defined by a second period of acquisition-time.

In the following description, reference is made to the accompanying figures, which show by way of illustration and examples how the invention may be practiced.

### EXAMPLE 1

### A Scanning System

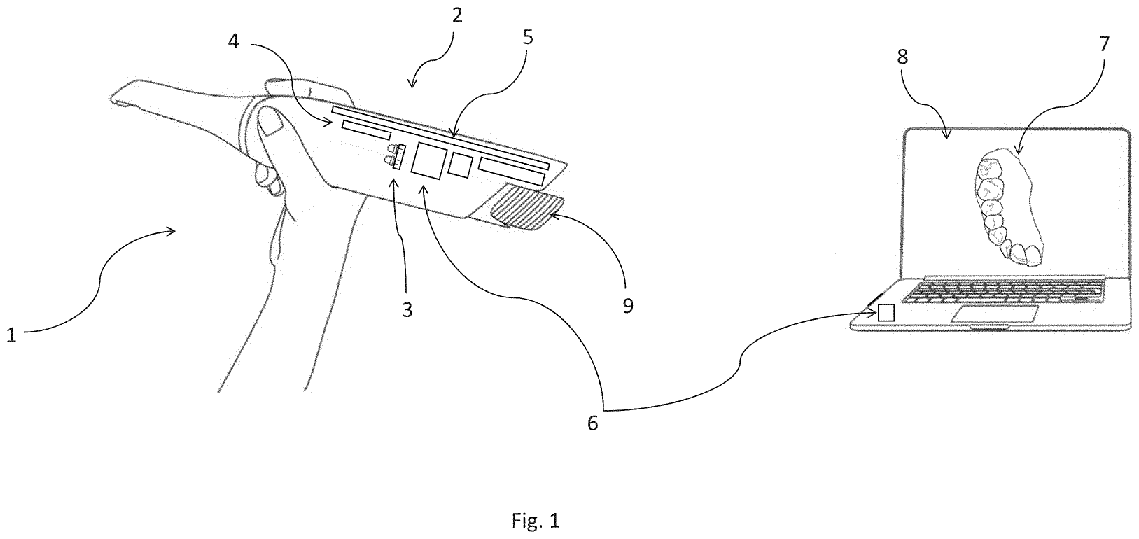

A scanning system according to the invention as disclosed herein is shown in FIG. 1.

FIG. 1 shows a scanning system 1 for determining a health-condition and/or a probability thereof based on scanning of an intraoral object, the scanning system 1 comprising: a scanning device 2 to scan the intraoral object, comprising: an illumination-unit 3 configured to illuminate the intraoral object with light; an image-sensor 4 configured to record images of light from the illuminated intraoral object; an illumination-controller 5 configured to operate the illumination-unit 3 in a first illumination-mode, and configured to operate the illumination unit 3 in a second illumination-mode, and/or an acquisition-controller 5 configured to operate the image-sensor 4 in a first acquisition-mode, and configured to operate the image-sensor 4 in a second acquisition-mode, wherein the scanning device 2 is configured to change between the first illumination-mode and the second illumination-mode, whereby the scanning device 2 forms a first dataset of the intraoral object when in the first illumination-mode and whereby the scanning device forms a second dataset of the intraoral object when in the second illumination-mode, and/or wherein the scanning device 2 is configured to change between the first acquisition-mode and the second acquisition-mode, whereby the scanning device forms a first dataset of the intraoral object when in the first acquisition-mode and whereby the scanning device 2 forms a second dataset of the intraoral object when in the second acquisition-mode; a data processor 6 configured to: form, from the first dataset, a 3D-model 7 of the intraoral object; form, from the second dataset, a 2D-image of the intraoral object and/or further details of the 3D-model; apply, on the 2D-image and/or the 3D-model 7, a diagnostic algorithm to identify a diagnostic feature of the intraoral object, determine, based on the diagnostic feature of the intraoral object, the health-condition and/or probability thereof.

In the first aspect of the invention, the data processor 6 is further configured to redefine, during the scan where the diagnostic feature is determined and based on the diagnostic feature or the related determined health-condition and/or the probability thereof, the first illumination-mode and the second illumination-mode, and/or redefine, during the scan where the diagnostic feature is determined and based on the diagnostic feature or the related determined health-condition and/or the probability thereof, the first acquisition-mode and the second acquisition-mode.

In the second aspect of the invention, the data processor 6 is further configured to define, based on a diagnostic training-feature of an intraoral object that differs from the diagnostic feature of the intraoral object, the first illumination-mode and the second illumination-mode, and/or define, based on a diagnostic training-feature of an intraoral object that differs from the diagnostic feature of the intraoral object, the first acquisition-mode and the second acquisition-mode.

In the fourth aspect of the invention, the acquisition-mode is defined by a first gain-value, and the second acquisition-mode is defined by a second gain-value.

In all aspects of the invention, the scanning system 1 further comprises a display 8, whereon the 3D-model 7 and the health-condition and/or the probability thereof are displayed.

Further details of the different aspects are shown and explained by the following examples.

### EXAMPLE 2

### Adaptive Scanning System According to the First Aspect

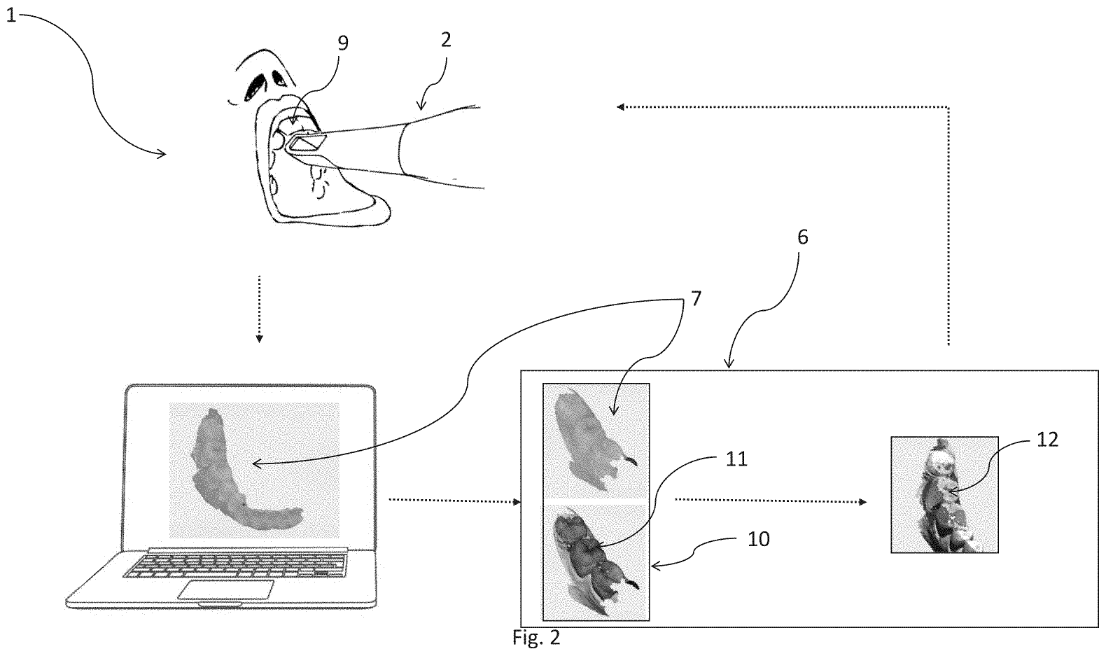

A scanning system according to the invention as disclosed herein is shown in FIG. 2.

FIG. 1 shows a scanning system 1 for determining a health-condition and/or a probability thereof based on scanning of an intraoral object 9.

As can be seen from FIG. 2, the data processor 6 forms firstly from the first dataset, a 3D-model 7 of the intraoral object 9. The data processor 6 forms secondly from the second dataset a 2D-image of the intraoral object and/or further details of the 3D-model. In this example, a plurality of 2D-images is formed, and parts of these 2D-images are then used to form an additional 3D-model 10. However, full 2D-images (i.e. 2D-images in color and with pixels corresponding to the sensor size) need not to be formed, and the second data set (for example readouts from only parts of the image sensor, and/or only a single color channel) can be used directly to form further details of the 3D-model.

In this example, the data processor 6 applies on the second data set, or part of a 2D-image (here being a part of the additional 3D-model 10) and/or the 3D-model 7, a diagnostic algorithm to identify a diagnostic feature 11 (here bacteria) of the intraoral object. The 2D-image or part(s) thereof may also be used to form further details of the 3D-model 7. For example, the 2D-image may provide fluorescence texture to the 3D-model 7, and the additional 3D-model 10 as shown (with fluorescence texture) may therefore be an updated 3D-model of the 3D-model 7 rather than an additional and separate 3D-model 10.

The data processor 6 further determines the health-condition 12 based on the diagnostic feature 11 (the bacteria and/or the density thereof) of the intraoral object 9. In this example, the health condition 12 is based on the fluorescence texture (from the updated 3D-model) and is a score for the bacteria density at the tooth/gingiva surface. The score is here associated with a color, here indicating that red is a high probability score, and yellow is a moderate score and no overlay color is normal score.

In the first aspect of the invention, as here exemplified, the data processor 6 redefines the first illumination-mode and the second illumination-mode. The redefinition, i.e. feedback to the scanning device 2, is made during the scan where the diagnostic feature 11 is determined and based on the diagnostic feature 11 or the related determined health-condition 12.

The example as here shown has two scanning modes—a white light illumination-mode and a fluorescence light illumination-mode.

The scanning device 2 thus changes between white light and blue light illumination. Every first image (with white light) comprise information associated with depth information and reflective color information. Every second image (with blue light) comprise the response of emitted fluorescence texture.

In this example, the scanning device 2 performs a scanning-session with 20 3D-frames per second (fps). Each 3D-frame comprises 90 2D-frames. Accordingly, the 20 3D-fps comprises 1800 2D-fps. Each 3D-frame may be acquired while a focus-lens is moved, for example in the time it takes the focus-lens to sweep a focus-distance.

Firstly, the scanning device 2 is set to acquire 16 3D-frames in white light-illumination, and 4 fluorescence 3D-frames in fluorescence light illumination in 1 second. For example, every fourth 3D-frame could be in fluorescence light illumination-mode. Thereby, the primary part of the acquired scan data is acquired using the white light illumination-mode. More specifically, the scanning device is defined to have 80/20 split ratio between 3D-frames obtained in the white light-illumination mode and the fluorescence light illumination-mode. During scanning, as shown in FIG. 2, the scanning device 2 redefines the first illumination-mode and the second illumination-mode, more specifically it changes the split ratio, this based on the determined health-condition 12.

Secondly, because there is a high probability score, the fluorescence light illumination-mode is increased, and the white light illumination-mode is decreased, for example to a split ratio of 20/80.

This redefinition provides that the scanning device 2 is now able to acquire high quality fluorescence data in the region of interest, where the score is high.

In addition to this example, the data processor 6 may redefine, during the scan where the diagnostic feature 11 is determined and based on the diagnostic feature 11 or the related determined health-condition 12, the first acquisition-mode and the second acquisition-mode. For example, a high-definition (HD) fluorescence 2D-image and/or HD reflective color image of the region may be acquired by redefining the acquisition modes.

The redefinition of said modes is during scanning.

The redefinition is here shown as based on data that is acquired during scanning, i.e. during the scan in which the diagnostic feature is determined.

However, the redefinition of said modes may be based on historical data that is acquired at previous scans, i.e. before the scan in which the diagnostic feature 11 is determined, for example 25 hours or more before the scan in which the diagnostic feature 11 is determined.

Regardless of what the redefinition is based upon, the redefinition of said modes is effectuated during scanning.

This may work in the following manner. The data processor registers and fuses incoming data together to construct a 3D model 7 of the dentition. A historical model of the same dentition is identified and aligned to the 3D-model. The historical 3D-model may comprise historical data acquired by different modes, such as IR data, fluorescence data, HD images, x-ray data and/or different types of annotations assigned to specific areas on the historical 3D-model.

In one example, the scanning system compares the topology of the present data and the historical model. If the distance between corresponding regions in present model and the historical model exceeds a threshold, the data processor 6 instructs the scanning device 2 to immediately acquire a HD color snapshot image of the identified region of interest.

In another example, the scanning system 2 compares in real-time the difference between historical fluorescence 3D-model and the present fluorescence 3D-model from the dentition. If a significant discrepancy between the historical 3D-model and the present 3D-model 7 is computed, the scanning device 2 is instructed to redefine, during scanning, the 3D frame rate split ratio.

### EXAMPLE 3

### Adaptive Scanning System According to the Fourth Aspect

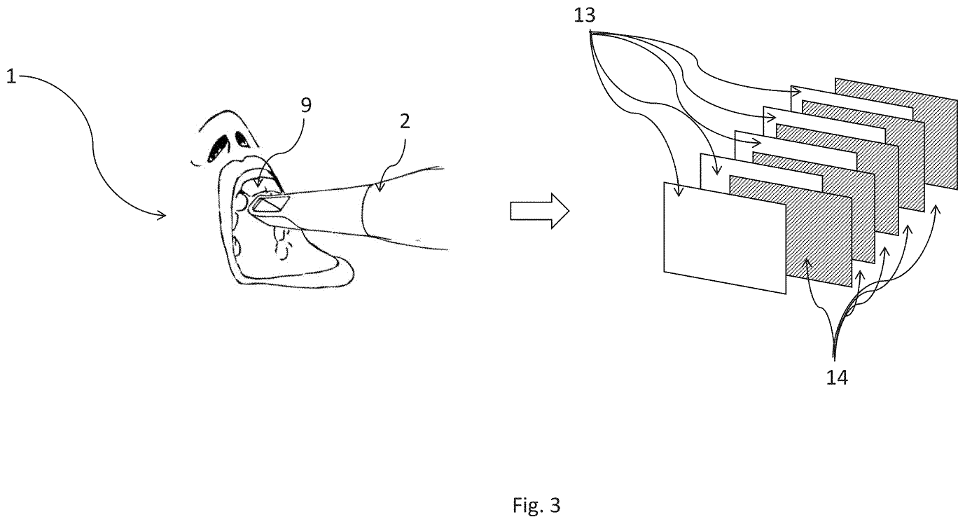

FIG. 3 shows a scanning system 1 for determining a health-condition and/or a probability thereof based on scanning of an intraoral object 9.

The scanning system 1 comprises: a scanning device 2 to scan the intraoral object, comprising: an illumination-unit 3 configured to illuminate the intraoral object with light; an image-sensor 4 configured to record images of light from the illuminated intraoral object; an illumination-controller 5 configured to operate the illumination-unit 3 in a first illumination-mode.

This first illumination is in this example a white-light illumination-mode. The scanning device is further configured to operate the illumination unit 3 in a second illumination-mode. This second illumination-mode is in this example a fluorescence-light illumination-mode.

The scanning device further comprises an acquisition-controller 5 configured to operate the image-sensor 4 in a first acquisition-mode and configured to operate the image-sensor 4 in a second acquisition-mode.

The scanning device 2 is configured to change between the first illumination-mode and the second illumination-mode, whereby the scanning device 2 forms a first dataset 13 of the intraoral object when in the first illumination-mode and whereby the scanning device forms a second dataset 14 of the intraoral object when in the second illumination-mode.

The scanning device 2 is further configured to change between the first acquisition-mode and the second acquisition-mode, whereby the scanning device forms the first dataset 13 of the intraoral object when in the first acquisition-mode and whereby the scanning device 2 forms the second dataset 14 of the intraoral object when in the second acquisition-mode.

The first data set 13 and the second data set 14 are acquired in an alternating manner as shown in FIG. 3. The first data set 13 to form the 3D-model 7 are different parts of 2D-images (the 2D-images shown in FIG. 3 as white frames). The second data set 14 is in its basis form one of the 2D-images, for example shown next to the white frames, or between the white frames, i.e. the gray frames in FIG. 3. However, the second data 14 may be a plurality of frames. The second data 14 is here used to form further details, here in the form of fluorescence texture, of the 3D-model.

The processor 6 then applies on the 3D-model 7, a diagnostic algorithm to identify a diagnostic feature of the intraoral object, and determines, based on the diagnostic feature of the intraoral object, the health-condition and/or probability thereof. Due to the gain being adjusted to each dataset, or to each image, and to each illumination-source, the health-condition and/or probability thereof is optimized.

The scanning device as exemplified herein comprises an LED die located in the back of the optical system to emit white light in the white-light illumination-mode. White light is passing through a polarizer before illuminated on to the object. Only light reflected directly form the surface of the object to be scanned will pass back to polarizer and impinge on the image sensor. The sensor acquires a series of 2D images while a focus lens is moved back and forth to generate a scan volume. The processor in the scanning device combines the 2D image stack into a 3D frame (called a sub-scan) which is transmitted to a scanning application software which constructs and render a 3D-model. More precisely, 2D-data from the image sensor is combined into a 3D-frame. The sensor operates at a 2D-framerate of between 50 and 200 images pr. sweep (one focus length travel distance) where the exposure time of the sensor is set by the sensor controller module, for example defined by a tic value between 5.000 and 50.000 tics.

The scanning device as exemplified herein further comprises two LED dies located in the back of the optical system inside the scanner to emit blue light at a wavelength of between 380 nm and 450 nm. The blue light is illuminated on to the object where a fraction of the light is inducing a fluorescence response from bacteria on the surface and. Most of the blue light is reflected from the surface. In order to isolate the fluorescence response from the surface, a long pass filter is mounted in front of the image sensor to filter out all blue light reflected from the surface. This allows only the tiny amount of the emitted fluorescence photons to access the sensor. Accordingly, the acquired signal is weak. The signal on the sensor is therefore electrically amplified by adjusting the sensor gain in this mode to two times that of the white-light illumination-mode.

### EXAMPLE 14

### Display of the 3D-Model and a Health-Condition

In all aspects of the invention, there is a display, whereon the 3D-model and the health-condition and/or the probability thereof are displayed.

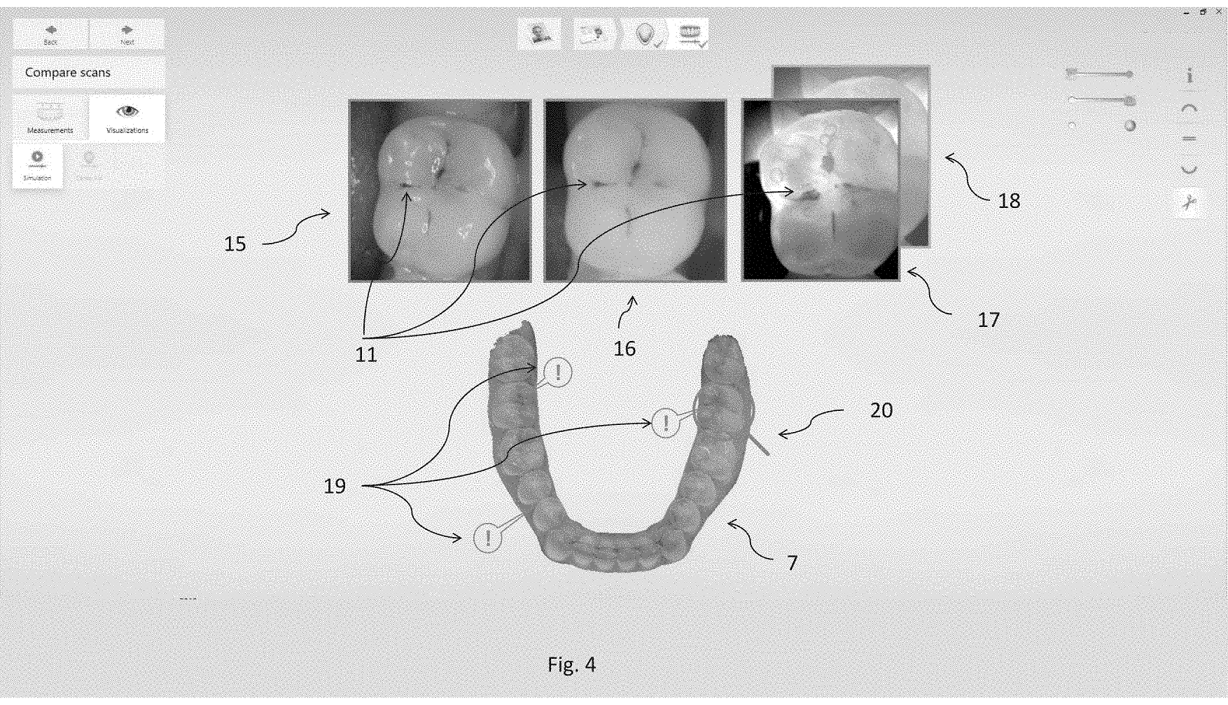

FIG. 4 shows an example of how the 3D-model 7 and the health-condition 12 are displayed. In this example, three health-conditions 12 will be displayed in relation to the 3D-model 7 once the operator of the display clicks on one of the here shown 3D-diagnosis indicators 19. The health-condition will appear in a pop-up screen and present the user with the actual health-condition, for example “carries”.

The health-condition is thus displayed with a 3D-diagnosis indicator between the health-condition and the 3D-model. The 3D-diagnosis indicator 19 shows how the health-condition (to pop-up) correlates to the 3D-location of the 3D-point on or inside the 3D-model 7 and helps the user to quickly navigate to the region of interest.

The exemplified user-interface further shows a high-definition 2D-image 15, a fluorescence 2D-image 16, and an infrared 2D-image 17.

All the 2D-images show a diagnostic feature 11. Further, each 2D-image is displayed with a 2D-3D indicator 20 between each of the 2D-images and the 3D-model to show how the 2D-feature 11 correlates to the 3D-location of the 3D-point on or inside the 3D-model 7. The 2D-3D indicator 20 is in this case shown as a magnifying glass. When the magnifying glass is moved to a location on the 3D-model, the one or more 2D-images appear on the display, so the user is able to also see the diagnostic feature 11 in 2D.

The 2D-images are recorded using the scanning system 1 according to the invention. Further, in this example, there also exist additional infrared 2D-images 18 that are acquired prior the scan. The operator is thus able to see how the carries has developed over time.

To provide an infrared 2D-image, the scanning device comprise a tip having a plurality of IR LED dies (840 nm). When scanning in IR mode the object to be analyzed is exposed to a mix of white light and IR light during the acquisition sequence. During one sweep, the sensor is acquiring white light images initially and at a specific position of the focus lens, the scanner switch off the white light and turns on the IR light while changing the sensor exposure time to 100.000 tics. When the IR image is acquired the scanner turns off the IR light and switches the white light illumination and decreases the 2D exposure time back to 20.000 tics while finishing the sweep. This result in a partial 3D frame and one IR 2D images acquired with a 5 times longer exposure time then the normal 2D white light images.

Further examples are described in further detail by the following items.

### Items:

1. A scanning system for determining a health-condition and/or a probability thereof based on scanning of an intraoral object, the scanning system comprising:
a scanning device to scan the intraoral object, comprising:
   an illumination-unit configured to illuminate the intraoral object with light;
   an image-sensor configured to record images of light from the illuminated intraoral object;
   an illumination-controller configured to operate the illumination-unit in a first illumination-mode, and configured to operate the illumination unit in a second illumination-mode, and/or
   an acquisition-controller configured to operate the image-sensor in a first acquisition-mode, and configured to operate the image-sensor in a second acquisition-mode,
   wherein the scanning device is configured to change between the first illumination-mode and the second illumination-mode, whereby the scanning device forms a first dataset of the intraoral object when in the first illumination-mode and whereby the scanning device forms a second dataset of the intraoral object when in the second illumination-mode, and/or
   wherein the scanning device is configured to change between the first acquisition-mode and the second acquisition-mode, whereby the scanning device forms a first dataset of the intraoral object when in the first acquisition-mode and whereby the scanning device forms a second dataset of the intraoral object when in the second acquisition-mode;
a data processor configured to:
   form, from the first dataset, a 3D-model of the intraoral object;
   form, from the second dataset, a 2D-image of the intraoral object and/or further details of the 3D-model;
   apply, on the 2D-image and/or the 3D-model, a diagnostic algorithm to identify a diagnostic feature of the intraoral object,
   determine, based on the diagnostic feature of the intraoral object, the health-condition and/or probability thereof,
   redefine, during the scan where the diagnostic feature is determined and based on the diagnostic feature or the related determined health-condition and/or the probability thereof, the first illumination-mode and the second illumination-mode, and/or
   redefine, during the scan where the diagnostic feature is determined and based on the diagnostic feature or the related determined health-condition and/or the probability thereof, the first acquisition-mode and the second acquisition-mode; and
a display, whereon the 3D-model and the health-condition and/or the probability thereof are displayed.

2. The scanning system according to item 1, wherein the determination of the health-condition and/or the probability thereof is independent of one or more dataset(s) of the intraoral object that is/are formed 24 hours or more before the first dataset and second dataset being formed.

3. The scanning system according to item 1, wherein the data processor is further configured to correlate at least a part of the 2D-image to at least a corresponding 3D-point on or inside the 3D-model, whereby the at least part of the 2D-image and the health-condition and/or the probability thereof is related to a 3D-location of the 3D-point on or inside the 3D-model.

4. The scanning system according to item 3, wherein the 2D-image or the at least part of the 2D-image with the diagnostic feature is displayed with a 2D-3D indicator between the 2D-image or the at least part of the 2D-image and the 3D-model to show how the 2D-feature correlates to the 3D-location of the 3D-point on or inside the 3D-model.

5. The scanning system according to any of the items 3-4, wherein the health-condition and/or the probability thereof is displayed with a 3D-diagnosis indicator between the health-condition and/or the probability thereof and the 3D-model to show how the health-condition and/or the probability thereof correlates to the 3D-location of the 3D-point on or inside the 3D-model.

6. The scanning system according to any of the items 3-5, wherein the health-condition and/or the probability thereof is displayed with a 2D-diagnosis indicator between the health-condition and/or the probability thereof and the 2D-image or the at least part of the 2D-image to show how the health-condition and/or the probability thereof correlates to the diagnostic feature.

7. The scanning system according to any of the preceding items, wherein the 2D-image or part(s) thereof is/are used to form further details of the 3D-model.

8. The scanning system according to any of the preceding items, wherein the health-condition and/or the probability thereof is related to carries and/or bacteria.

9. The scanning system according to any of the preceding items, wherein the health-condition and/or the probability thereof is related to cancer.

10. The scanning system according to any of the preceding items, wherein the diagnostic algorithm is based on artificial intelligence and/or is based on pattern recognition.

11. The scanning system according to any of the preceding items, wherein said first illumination mode is white-light-illumination, and wherein said second illumination mode is fluorescence-light-illumination.

12. The scanning system according to any of the preceding items, wherein said first illumination mode is white-light-illumination, and wherein said second illumination mode is infrared-light-illumination.

13. The scanning system according to any of the preceding items, wherein said first acquisition-mode is defined by a low spatial resolution and said second acquisition-mode is defined by a high spatial resolution.

14. The scanning system according to any of the preceding items, wherein said first acquisition-mode is defined by a first gain-value and said second acquisition-mode is defined by a second gain-value.

15. The scanning system according to any of the preceding items, wherein said first illumination-mode is defined by a first period of illumination-time and said second illumination-mode is defined by a second period of illumination-time.

16. The scanning system according to any of the preceding items, wherein said first acquisition-mode is defined by a first period of acquisition-time and said second acquisition-mode is defined by a second period of acquisition-time.

17. A scanning system for determining a health-condition and/or a probability thereof based on scanning of an intraoral object, the scanning system comprising:
a scanning device to scan the intraoral object, comprising:
   an illumination-unit configured to illuminate the intraoral object with light;
   an image-sensor configured to record images of light from the illuminated intraoral object;
   an illumination-controller configured to operate the illumination-unit in a first illumination-mode, and configured to operate the illumination unit in a second illumination-mode, and/or
   an acquisition-controller configured to operate the image-sensor in a first acquisition-mode, and configured to operate the image-sensor in a second acquisition-mode,
   wherein the scanning device is configured to change between the first illumination-mode and the second illumination-mode, whereby the scanning device forms a first dataset of the intraoral object when in the first illumination-mode and whereby the scanning device forms a second dataset of the intraoral object when in the second illumination-mode, and/or
   wherein the scanning device is configured to change between the first acquisition-mode and the second acquisition-mode, whereby the scanning device forms a first dataset of the intraoral object when in the first acquisition-mode and whereby the scanning device forms a second dataset of the intraoral object when in the second acquisition-mode;
a data processor configured to:
   form, from the first dataset, a 3D-model of the intraoral object;
   form, from the second dataset, a 2D-image of the intraoral object and/or further details of the 3D-model;
   apply, on the 2D-image and/or the 3D-model, a diagnostic algorithm to identify a diagnostic feature of the intraoral object,
   determine, based on the diagnostic feature of the intraoral object, the health-condition and/or probability thereof,
   define, based on a diagnostic training-feature of an intraoral object that differs from the diagnostic feature of the intraoral object, the first illumination-mode and the second illumination-mode, and/or
   define, based on a diagnostic training-feature of an intraoral object that differs from the diagnostic feature of the intraoral object, the first acquisition-mode and the second acquisition-mode; and
a display, whereon the 3D-model and the health-condition and/or probability thereof are displayed.

18. The scanning system according to item 17, wherein the determination of the health-condition and/or the probability thereof is independent of one or more dataset(s) of the intraoral object that is/are formed 24 hours or more before the first dataset and second dataset being formed.

19. The scanning system according to any of the items 17-19, wherein the data processor is further configured to correlate at least a part of the 2D-image to at least a corresponding 3D-point on or inside the 3D-model, whereby the at least part of the 2D-image and the health-condition and/or the probability thereof is related to a 3D-location of the 3D-point on or inside the 3D-model.

20. The scanning system according to item 19, wherein the 2D-image or the at least part of the 2D-image with the diagnostic feature is displayed with a 2D-3D indicator between the 2D-image or the at least part of the 2D-image and the 3D-model to show how the 2D-feature correlates to the 3D-location of the 3D-point on or inside the 3D-model.

21. The scanning system according to any of the items 19-20, wherein the health-condition and/or the probability thereof is displayed with a 3D-diagnosis indicator between the health-condition and/or the probability thereof and the 3D-model to show how the health-condition and/or the probability thereof correlates to the 3D-location of the 3D-point on or inside the 3D-model.

22. The scanning system according to any of the items 19-21, wherein the health-condition and/or the probability thereof is displayed with a 2D-diagnosis indicator between the health-condition and/or the probability thereof and the 2D-image or the at least part of the 2D-image to show how the health-condition and/or the probability thereof correlates to the diagnostic feature.

23. The scanning system according to any of the items 17-22, wherein the 2D-image or part(s) thereof is/are used to form further details of the 3D-model.

24. The scanning system according to any of the items 17-23, wherein the health-condition and/or the probability thereof is related to carries and/or bacteria.

25. The scanning system according to any of the items 17-24, wherein the health-condition and/or the probability thereof is related to cancer.

26. The scanning system according to any of the items 17-25, wherein the diagnostic algorithm is based on artificial intelligence and/or is based on pattern recognition.

27. The scanning system according to any of the items 17-26, wherein said first illumination mode is white-light-illumination, and wherein said second illumination mode is fluorescence-light-illumination.

28. The scanning system according to any of the items 17-27, wherein said first illumination mode is white-light-illumination, and wherein said second illumination mode is infrared-light-illumination.

29. The scanning system according to any of the items 17-28, wherein said first acquisition-mode is defined by a low spatial resolution and said second acquisition-mode is defined by a high spatial resolution.

30. The scanning system according to any of the items 17-29, wherein said first acquisition-mode is defined by a first gain-value and said second acquisition-mode is defined by a second gain-value.

31. The scanning system according to any of the items 17-30, wherein said first illumination-mode is defined by a first period of illumination-time and said second illumination-mode is defined by a second period of illumination-time.

32. The scanning system according to any of the items 17-31, wherein said first acquisition-mode is defined by a first period of acquisition-time and said second acquisition-mode is defined by a second period of acquisition-time.

33. A scanning system for determining a health-condition and/or a probability thereof based on scanning of an intraoral object, the scanning system comprising:
a scanning device to scan the intraoral object, comprising:
   an illumination-unit configured to illuminate the intraoral object with light;
   an image-sensor configured to record images of light from the illuminated intraoral object;
   an illumination-controller configured to operate the illumination-unit in a first illumination-mode, and configured to operate the illumination unit in a second illumination-mode, and/or
   an acquisition-controller configured to operate the image-sensor in a first acquisition-mode, and configured to operate the image-sensor in a second acquisition-mode,
   wherein the scanning device is configured to change between the first illumination-mode and the second illumination-mode, whereby the scanning device forms a first dataset of the intraoral object when in the first illumination-mode and whereby the scanning device forms a second dataset of the intraoral object when in the second illumination-mode, and/or
   wherein the scanning device is configured to change between the first acquisition-mode and the second acquisition-mode, whereby the scanning device forms a first dataset of the intraoral object when in the first acquisition-mode and whereby the scanning device forms a second dataset of the intraoral object when in the second acquisition-mode;
a data processor configured to:
   form, from the first dataset, a 3D-model of the intraoral object;
   form, from the second dataset, a 2D-image of the intraoral object and/or further details of the 3D-model;
   apply, on the 2D-image and/or the 3D-model, a diagnostic algorithm to identify a diagnostic feature of the intraoral object,
   determine, based on the diagnostic feature of the intraoral object, the health-condition and/or probability thereof,
   redefine, during the scan where the diagnostic feature is determined and based on the diagnostic feature or the related determined health-condition and/or the probability thereof, the first illumination-mode and the second illumination-mode, and/or
   redefine, during the scan where the diagnostic feature is determined and based on the diagnostic feature or the related determined health-condition and/or the probability thereof, the first acquisition-mode and the second acquisition-mode, and/or
   define, based on a diagnostic training-feature of an intraoral object that differs from the diagnostic feature of the intraoral object, the first illumination-mode and the second illumination-mode, and/or
   define, based on a diagnostic training-feature of an intraoral object that differs from the diagnostic feature of the intraoral object, the first acquisition-mode and the second acquisition-mode; and
a display, whereon the 3D-model and the health-condition and/or probability thereof are displayed.

34. The scanning system according to item 33, wherein the determination of the health-condition and/or the probability thereof is independent of one or more dataset(s) of the intraoral object that is/are formed 24 hours or more before the first dataset and second dataset being formed.

35. The scanning system according to any of the items 33-34, The data processor is further configured to correlate at least a part of the 2D-image to at least a corresponding 3D-point on or inside the 3D-model, whereby the at least part of the 2D-image and the health-condition and/or the probability thereof is related to a 3D-location of the 3D-point on or inside the 3D-model.

36. The scanning system according to item 35, wherein the 2D-image or the at least part of the 2D-image with the diagnostic feature is displayed with a 2D-3D indicator between the 2D-image or the at least part of the 2D-image and the 3D-model to show how the 2D-feature correlates to the 3D-location of the 3D-point on or inside the 3D-model.

37. The scanning system according to any of the items 35-36, wherein the health-condition and/or the probability thereof is displayed with a 3D-diagnosis indicator between the health-condition and/or the probability thereof and the 3D-model to show how the health-condition and/or the probability thereof correlates to the 3D-location of the 3D-point on or inside the 3D-model.

38. The scanning system according to any of the items 35-37, wherein the health-condition and/or the probability thereof is displayed with a 2D-diagnosis indicator between the health-condition and/or the probability thereof and the 2D-image or the at least part of the 2D-image to show how the health-condition and/or the probability thereof correlates to the diagnostic feature.

39. The scanning system according to any of the items 33-38, wherein the 2D-image or part(s) thereof is/are used to form further details of the 3D-model.

40. The scanning system according to any of the items 33-39, wherein the health-condition and/or the probability thereof is related to carries and/or bacteria.

41. The scanning system according to any of the items 33-40, wherein the health-condition and/or the probability thereof is related to cancer.

42. The scanning system according to any of the items 33-41, wherein the diagnostic algorithm is based on artificial intelligence and/or is based on pattern recognition.

43. The scanning system according to any of the items 33-42, wherein said first illumination mode is white-light-illumination, and wherein said second illumination mode is fluorescence-light-illumination.

44. The scanning system according to any of the items 33-43, wherein said first illumination mode is white-light-illumination, and wherein said second illumination mode is infrared-light-illumination.

45. The scanning system according to any of the items 33-44, wherein said first acquisition-mode is defined by a low spatial resolution and said second acquisition-mode is defined by a high spatial resolution.

46. The scanning system according to any of the items 33-45, wherein said first acquisition-mode is defined by a first gain-value and said second acquisition-mode is defined by a second gain-value.

47. The scanning system according to any of the items 33-46, wherein said first illumination-mode is defined by a first period of illumination-time and said second illumination-mode is defined by a second period of illumination-time.

48. The scanning system according to any of the items 33-47, wherein said first acquisition-mode is defined by a first period of acquisition-time and said second acquisition-mode is defined by a second period of acquisition-time.

49. A scanning system for determining a health-condition and/or a probability thereof based on scanning of an intraoral object, the scanning system comprising:
a scanning device to scan the intraoral object, comprising:
   an illumination-unit configured to illuminate the intraoral object with light;
   an image-sensor configured to record images of light from the illuminated intraoral object;
   an illumination-controller configured to operate the illumination-unit in a first illumination-mode, and configured to operate the illumination unit in a second illumination-mode; and
   an acquisition-controller configured to operate the image-sensor in a first acquisition-mode, wherein the first acquisition-mode is defined by a first gain-value, and configured to operate the image-sensor in a second acquisition-mode, wherein the second acquisition-mode is defined by a second gain-value,
   wherein the scanning device is configured to change between the first illumination-mode and the second illumination-mode, whereby the scanning device forms a first dataset of the intraoral object when in the first illumination-mode and whereby the scanning device forms a second dataset of the intraoral object when in the second illumination-mode, and
   wherein the scanning device is further configured to change between the first acquisition-mode and the second acquisition-mode, whereby the scanning device forms the first dataset of the intraoral object when in the first acquisition-mode and whereby the scanning device forms the second dataset of the intraoral object when in the second acquisition-mode;
a data processor configured to:
   form, from the first dataset, a 3D-model of the intraoral object;
   form, from the second dataset, a 2D-image of the intraoral object and/or further details of the 3D-model;
   apply, on the 2D-image and/or the 3D-model, a diagnostic algorithm to identify a diagnostic feature of the intraoral object,
   determine, based on the diagnostic feature of the intraoral object, the health-condition and/or probability thereof,
a display, whereon the 3D-model and the health-condition and/or probability thereof are displayed.

50. The scanning system according to item 49, wherein the determination of the health-condition and/or the probability thereof is independent of one or more dataset(s) of the intraoral object that is/are formed 24 hours or more before the first dataset and second dataset being formed.

51. Scanning system according to any of the items 49-50, wherein the data processor is further configured to correlate at least a part of the 2D-image to at least a corresponding 3D-point on or inside the 3D-model, whereby the at least part of the 2D-image and the health-condition and/or the probability thereof is related to a 3D-location of the 3D-point on or inside the 3D-model.

52. The scanning system according to item 51, wherein the 2D-image or the at least part of the 2D-image with the diagnostic feature is displayed with a 2D-3D indicator between the 2D-image or the at least part of the 2D-image and the 3D-model to show how the 2D-feature correlates to the 3D-location of the 3D-point on or inside the 3D-model.

53. The scanning system according to any of the items 51-52, wherein the health-condition and/or the probability thereof is displayed with a 3D-diagnosis indicator between the health-condition and/or the probability thereof and the 3D-model to show how the health-condition and/or the probability thereof correlates to the 3D-location of the 3D-point on or inside the 3D-model.

54. The scanning system according to any of the items 51-53, wherein the health-condition and/or the probability thereof is displayed with a 2D-diagnosis indicator between the health-condition and/or the probability thereof and the 2D-image or the at least part of the 2D-image to show how the health-condition and/or the probability thereof correlates to the diagnostic feature.

55. The scanning system according to any of the items 49-54, wherein the 2D-image or part(s) thereof is/are used to form further details of the 3D-model.

56. The scanning system according to any of the items 49-55, wherein the health-condition and/or the probability thereof is related to carries and/or bacteria.

57. The scanning system according to any of the items 49-56, wherein the health-condition and/or the probability thereof is related to cancer.

58. The scanning system according to any of the items 49-57, wherein the diagnostic algorithm is based on artificial intelligence and/or is based on pattern recognition.

59. The scanning system according to any of the preceding 49-58, wherein said first illumination mode is white-light-illumination, and wherein said second illumination mode is fluorescence-light-illumination.

60. The scanning system according to any of the items 49-59, wherein said first illumination mode is white-light-illumination, and wherein said second illumination mode is infrared-light-illumination.

61. The scanning system according to any of the items 49-60, wherein said first acquisition-mode is further defined by a low spatial resolution and said second acquisition-mode is further defined by a high spatial resolution.

62. The scanning system according to any of the items 49-61, wherein said first illumination-mode is defined by a first period of illumination-time and said second illumination-mode is defined by a second period of illumination-time.

63. The scanning system according to any of the items 49-62, wherein said first acquisition-mode is further defined by a first period of acquisition-time and said second acquisition-mode is further defined by a second period of acquisition-time.

64. The scanning system according to any of the items, wherein the scanning device is powered by an internal power supply, preferably a battery.

65. The scanning system according to any of the items 49-64, wherein the first gain-value and the second gain-value are controlled via a pin to the image sensor, whereby the first gain-value is synchronized with the first dataset or parts thereof, and the second gain-value is synchronized with the second dataset or parts thereof or the 2D-image or parts thereof.

1. A scanning system for determining a health-condition and/or a probability thereof based on scanning of an intraoral object, the scanning system comprising:
a scanning device to scan the intraoral object, comprising:
an illumination-unit configured to illuminate the intraoral object with light;
an image-sensor configured to record images of light from the illuminated intraoral object;
an illumination-controller configured to operate the illumination-unit in a first illumination-mode, and configured to operate the illumination unit in a second illumination-mode, and/or
an acquisition-controller configured to operate the image-sensor in a first acquisition-mode, and configured to operate the image-sensor in a second acquisition-mode, wherein the scanning device is configured to change between the first illumination-mode and the second illumination-mode, whereby the scanning device forms a first dataset of the intraoral object when in the first illumination-mode and whereby the scanning device forms a second dataset of the intraoral object when in the second illumination-mode, and/or
wherein the scanning device is configured to change between the first acquisition-mode and the second acquisition-mode, whereby the scanning device forms a first dataset of the intraoral object when in the first acquisition-mode and whereby the scanning device forms a second dataset of the intraoral object when in the second acquisition-mode;
a data processor configured to:
form, from the first dataset, a 3D-model of the intraoral object;
form, from the second dataset, a 2D-image of the intraoral object and/or further details of the 3D-model;
apply, on the 2D-image and/or the 3D-model, a diagnostic algorithm to identify a diagnostic feature of the intraoral object,
determine, based on the diagnostic feature of the intraoral object, the health-condition and/or probability thereof,
redefine, during the scan where the diagnostic feature is determined and based on the diagnostic feature or the related determined health-condition and/or the probability thereof, the first illumination-mode and the second illumination-mode, and/or
redefine, during the scan where the diagnostic feature is determined and based on the diagnostic feature or the related determined health-condition and/or the probability thereof, the first acquisition-mode and the second acquisition-mode; and
a display, whereon the 3D-model and the health-condition and/or the probability thereof are displayed.

2. The scanning system according to claim 1, wherein the determination of the health-condition and/or the probability thereof is independent of one or more dataset(s) of the intraoral object that is/are formed 24 hours or more before the first dataset and second dataset being formed.

3. The scanning system according to claim 1, wherein the data processor is further configured to correlate at least a part of the 2D-image to at least a corresponding 3D-point on or inside the 3D-model, whereby the at least part of the 2D-image and the health-condition and/or the probability thereof is related to a 3D-location of the 3D-point on or inside the 3D-model.

4. The scanning system according to claim 3, wherein the 2D-image or the at least part of the 2D-image with the diagnostic feature is displayed with a 2D-3D indicator between the 2D-image or the at least part of the 2D-image and the 3D-model to show how the 2D-feature correlates to the 3D-location of the 3D-point on or inside the 3D-model.

5. The scanning system according to claim 3, wherein the health-condition and/or the probability thereof is displayed with a 3D-diagnosis indicator between the health-condition and/or the probability thereof and the 3D-model to show how the health-condition and/or the probability thereof correlates to the 3D-location of the 3D-point on or inside the 3D-model.

6. The scanning system according to claim 3, wherein the health-condition and/or the probability thereof is displayed with a 2D-diagnosis indicator between the health-condition and/or the probability thereof and the 2D-image or the at least part of the 2D-image to show how the health-condition and/or the probability thereof correlates to the diagnostic feature.

7. The scanning system according to claim 1, wherein the diagnostic algorithm is based on artificial intelligence and/or is based on pattern recognition.

8. The scanning system according to claim 1, wherein said first acquisition-mode is defined by a first gain-value and said second acquisition-mode is defined by a second gain-value.

9. The scanning system according to claim 1, wherein said first illumination-mode is defined by a first period of illumination-time and said second illumination-mode is defined by a second period of illumination-time.

10. The scanning system according to claim 1, wherein said first acquisition-mode is defined by a first period of acquisition-time and said second acquisition-mode is defined by a second period of acquisition-time.

11. A scanning system for determining a health-condition and/or a probability thereof based on scanning of an intraoral object, the scanning system comprising:
a scanning device to scan the intraoral object, comprising:
an illumination-unit configured to illuminate the intraoral object with light;
an image-sensor configured to record images of light from the illuminated intraoral object;
an illumination-controller configured to operate the illumination-unit in a first illumination-mode, and configured to operate the illumination unit in a second illumination-mode, and/or
an acquisition-controller configured to operate the image-sensor in a first acquisition-mode, and configured to operate the image-sensor in a second acquisition-mode, wherein the scanning device is configured to change between the first illumination-mode and the second illumination-mode, whereby the scanning device forms a first dataset of the intraoral object when in the first illumination-mode and whereby the scanning device forms a second dataset of the intraoral object when in the second illumination-mode, and/or
wherein the scanning device is configured to change between the first acquisition-mode and the second acquisition-mode, whereby the scanning device forms a first dataset of the intraoral object when in the first acquisition-mode and whereby the scanning device forms a second dataset of the intraoral object when in the second acquisition-mode;
a data processor configured to:
form, from the first dataset, a 3D-model of the intraoral object;
form, from the second dataset, a 2D-image of the intraoral object and/or further details of the 3D-model;
apply, on the 2D-image and/or the 3D-model, a diagnostic algorithm to identify a diagnostic feature of the intraoral object,
determine, based on the diagnostic feature of the intraoral object, the health-condition and/or probability thereof,
define, based on a diagnostic training-feature of an intraoral object that differs from the diagnostic feature of the intraoral object, the first illumination-mode and the second illumination-mode, and/or
define, based on a diagnostic training-feature of an intraoral object that differs from the diagnostic feature of the intraoral object, the first acquisition-mode and the second acquisition-mode; and
a display, whereon the 3D-model and the health-condition and/or probability thereof are displayed.

12. The scanning system according to claim 1, wherein said first acquisition-mode is defined by a first gain-value and said second acquisition-mode is defined by a second gain-value.

13. The scanning system according to claim 1, wherein said first illumination-mode is defined by a first period of illumination-time and said second illumination-mode is defined by a second period of illumination-time.

14. The scanning system according to claim 1, wherein said first acquisition-mode is defined by a first period of acquisition-time and said second acquisition-mode is defined by a second period of acquisition-time.

15. A scanning system for determining a health-condition and/or a probability thereof based on scanning of an intraoral object, the scanning system comprising:
a scanning device to scan the intraoral object, comprising:
an illumination-unit configured to illuminate the intraoral object with light;
an image-sensor configured to record images of light from the illuminated intraoral object;
an illumination-controller configured to operate the illumination-unit in a first illumination-mode, and configured to operate the illumination unit in a second illumination-mode; and
an acquisition-controller configured to operate the image-sensor in a first acquisition-mode, wherein the first acquisition-mode is defined by a first gain-value, and configured to operate the image-sensor in a second acquisition-mode, wherein the second acquisition-mode is defined by a second gain-value,
wherein the scanning device is configured to change between the first illumination-mode and the second illumination-mode, whereby the scanning device forms a first dataset of the intraoral object when in the first illumination-mode and whereby the scanning device forms a second dataset of the intraoral object when in the second illumination-mode, and
wherein the scanning device is further configured to change between the first acquisition-mode and the second acquisition-mode, whereby the scanning device forms the first dataset of the intraoral object when in the first acquisition-mode and whereby the scanning device forms the second dataset of the intraoral object when in the second acquisition-mode;
a data processor configured to:
form, from the first dataset, a 3D-model of the intraoral object;
form, from the second dataset, a 2D-image of the intraoral object and/or further details of the 3D-model;
apply, on the 2D-image and/or the 3D-model, a diagnostic algorithm to identify a diagnostic feature of the intraoral object,
determine, based on the diagnostic feature of the intraoral object, the health-condition and/or probability thereof,
a display, whereon the 3D-model and the health-condition and/or probability thereof are displayed.

16. The scanning system according to claim 15, wherein the scanning device is powered by an internal power supply, preferably a battery.

17. The scanning system according to claim 15, wherein the first gain-value and the second gain-value are controlled via a pin to the image sensor, whereby the first gain-value is synchronized with the first dataset or parts thereof, and the second gain-value is synchronized with the second dataset or parts thereof or the 2D-image or parts thereof.
